(19) United States
(12) Patent Application Publication
(10) Pub. No.: US 2012/0121458 A1
(43) Pub. Date: May 17, 2012

(54) METHODS FOR ERADICATING BEDBUGS USING ULTRAVIOLET C AND PHOTO CATALYTIC OXIDATION

(76) Inventor: Mohamed H. Tarifi, Derby, CT (US)

(21) Appl. No.: 13/244,107

(22) Filed: Sep. 23, 2011

Related U.S. Application Data
(60) Provisional application No. 61/414,099, filed on Nov. 16, 2010.

(51) Int. Cl. A61L 2/10

(52) U.S. Cl. 422/24

(57) ABSTRACT

Methods for eradicating bedbugs through the combined use of ultraviolet C (UVC) photons and photo catalytic oxidation (PCO) include spraying infested areas with titanium dioxide (TiO₂), applying UVC to activate the TiO₂ and trigger the PCO process. The PCO process may oxidize organic matter, which is abundantly available in infested articles, to carbon dioxide (CO₂) and water. The CO₂, along with carrying out the process in a dark environment, may attract bedbugs and force them to leave their hiding places, where the combination of UVC and PCO can destroy the bedbugs. A vacuum cleaner may be used to remove all bedbugs and render the infested area free of bedbugs. Typically, bed sheets should be washed and mattresses and couches should be steam-cleaned following bedbug eradication.

### CROSS REFERENCE TO RELATED APPLICATIONS

This application claims the benefit of priority of U.S. provisional patent application No. 61/414,099, filed Nov. 16, 2010, the contents of which are herein incorporated by reference.

### BACKGROUND OF THE INVENTION

The present invention relates to methods for eradicating bedbugs and, more particularly, to methods for eradicating bedbugs using ultraviolet C (UVC) and photo catalytic oxidation (PCO).

There have been attempts to control bedbug infestation through applications of insecticidal chemicals to infected areas and materials (especially mattresses). This approach has many drawbacks. For example, it can expose those using a treated area or mattress too soon after application to odor or other undesired toxic characteristics of the pesticidal chemical. Further, unless the chemicals are used regularly, without regard to whether an infestation is known to already exist (which procedure will significantly increase costs), those sleeping in an infected area can be bitten before one knows to begin treatment. Moreover, bedbugs have become resistant to pesticides, including DDT and organophosphates. Bedbugs have also developed resistance to pyrethoid insecticides as well. Hot water (at least 120° F.) may be successful for killing bedbugs; however, such a method is not practical for couches, mattress, and other infested items that would not fit into a washer.

Bedbugs feed on human blood. Thus, they are not merely unsightly, they leave ugly skin markings. However problematic this is for residential bedrooms, it is an even more serious problem for motels and the like. With respect to such commercial bedrooms there is more opportunity for external infection sources to bring bedbugs to the site, and should there be an unknown infestation which causes biting of customers before it is dealt with, there is a severe risk of customer dissatisfaction and adverse publicity, likely leading to a long term significant reputation and business loss.

As can be seen, there is a need for improved apparatus for controlling and eradicating bedbugs without the use of pesticides or other insecticidal chemicals.

### SUMMARY OF THE INVENTION

In one aspect of the present invention, a method for removing bedbugs comprises applying titanium dioxide to an area to be rendered free of bedbugs; and irradiating ultraviolet C (UVC) photons onto the area to be cleaned, the UVC photons triggering a photo catalytic oxidation (PCO) process that, along with UVC, would eradicate bedbugs by destroying its DNA.

In another aspect of the present invention, a method for removing bedbugs comprises applying titanium dioxide to an area to be cleaned; irradiating ultraviolet C (UVC) photons onto the area to be cleaned, the UVC photons initiating a photo catalytic oxidation (PCO) process that would breakdown abundantly available organic matter and oxidizing it to carbon dioxide and water. Bedbugs are known to be attracted to carbon dioxide which forces the bedbugs to come out of their hiding placed to be eradicated by both UVC photons and PCO.

In a further aspect of the present invention, a method for cleaning surfaces comprises applying titanium dioxide to an area to be cleaned; irradiating ultraviolet C (UVC) photons onto the area to be cleaned, the UVC photons initiating a photo catalytic oxidation (PCO) process to kill bedbugs; and vacuuming the area to be cleaned.

These and other features, aspects and advantages of the present invention will become better understood with reference to the following drawings, description and claims.

### BRIEF DESCRIPTION OF THE DRAWINGS

FIG. 1 is a perspective view of an apparatus for eradicating bedbugs, according to an exemplary embodiment of the present invention;

FIG. 2 is a bottom view of the apparatus of FIG. 1; and

FIG. 3 is a flow chart showing a method according to an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features.

Broadly, an embodiment of the present invention provides methods for eradicating bedbugs through the combined use of ultraviolet C (UVC) photons and photo catalytic oxidation (PCO). The methods include spraying infested areas with titanium dioxide (TiO₂), applying UVC to activate the TiO₂ and trigger the PCO process. The PCO process may oxidize organic matter to carbon dioxide (CO₂) and water. The CO₂ may attract bedbugs and force them to leave their hiding places, where the combination of UVC and PCO can destroy the bedbugs. A vacuum cleaner may be used to remove all bedbugs and render the infested area free of bedbugs.

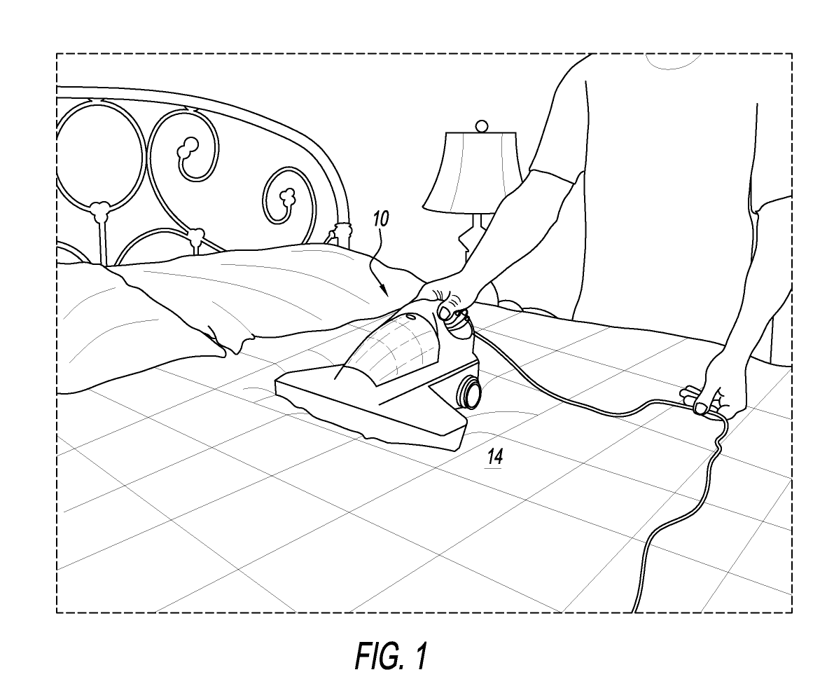

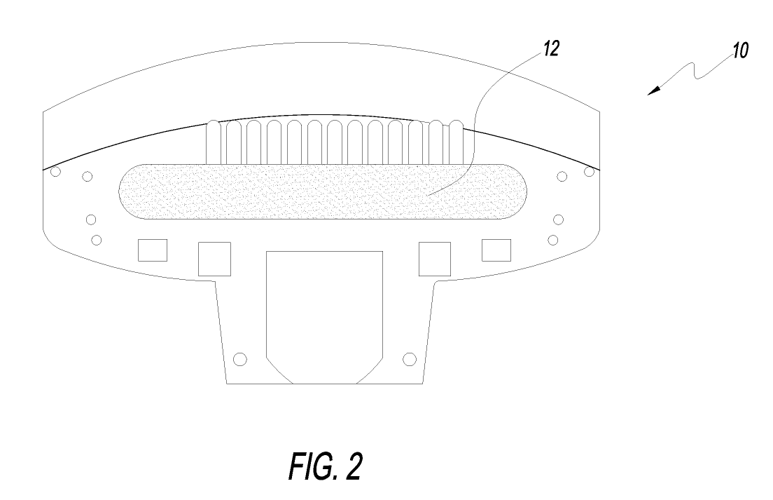

Referring to FIGS. 1 and 2, a device 10 may be a combined hand-held vacuum cleaner having a UVC lamp 12. The device 10 may have switches (not shown) to independently control the vacuum cleaner and the UVC lamp 12.

While FIG. 1 shows a corded device 10, the device 10 may be cordless, powered by rechargeable batteries, for example. While FIG. 1 shows a device 10 having an integrated vacuum and UVC lamp 12, in some embodiments, the UVC lamp may be separate from the vacuum. For example, the UVC may be applied via a hand-held UVC wand (not shown) and the vacuum may be a conventional vacuum cleaner. The device 10 may be disposed on an area to be cleaned 14, as described below.

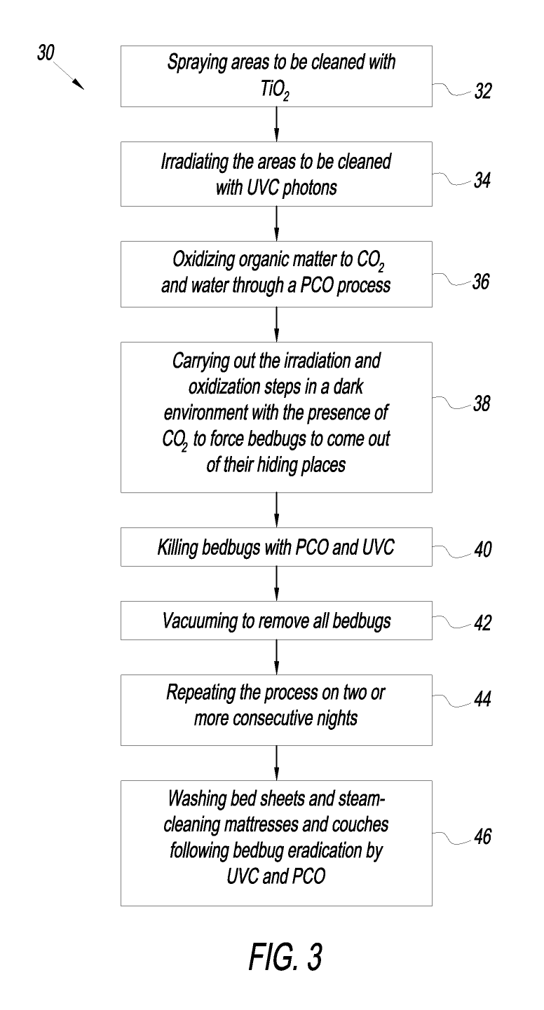

Referring to FIG. 3, a method 30 for eradicating bedbugs may include an initial step 32 of spraying areas to be cleaned with TiO₂. TiO₂ is safe and inert and may be used to turn the areas to be cleaned into a semi-conductor. A further step 34 may include irradiating the areas to be cleaned with UVC photons. The UVC may be supplied from a wand, a vacuum device 10 as shown in FIGS. 1 and 2, or some other UVC lamp. In a step 36, organic matter may be oxidized to CO₂ through a PCO process. The UVC photons, impinging on the semi-conductive surface to be cleaned, may activate the TiO₂ to generate hydroxyl radicals which may kill off bedbugs by destroying its DNA. In step 38, the irradiation and oxidization steps may be carried out in a dark environment with the presence of CO₂ to force the bedbugs to come out of their hiding places. In step 40, the bedbugs may be killed with PCO and UVC. In a further step 42, a vacuum cleaner may be used to remove all bedbugs and render the previously infested area to be free of bedbugs. In a further step 44, the process may be repeated on two or more consecutive nights to ensure total eradication has been achieved. Finally, in a further step 46, typically, bed sheets should be washed and mattresses and couches should be steam-cleaned following bedbug eradication by UVC and PCO, as described above.

While the above description describes using TiO₂ for the PCO process, other chemical combinations may be useful, such as binary oxides selected from TiO₂/SiO₂, TiO₂/ZrO₂, TiO₂/SnO₂, TiO₂/WO₃, TiO₂/MoO₃ and mixtures thereof.

The methods of the present invention may be used as a stand-alone method for the safe and effective removal of bedbugs. Further, the methods of the present invention may be used in conjunction with other bedbug removal methods that are known in the art.

It should be understood, of course, that the foregoing relates to exemplary embodiments of the invention and that modifications may be made without departing from the spirit and scope of the invention as set forth in the following claims.

What is claimed is:

1. A method for removing bedbugs, comprising:
applying titanium dioxide to an area to be cleaned; and
irradiating ultraviolet C (UVC) photons onto the area to be cleaned, the UVC photons triggering a photo catalytic oxidation (PCO) process that would, in combination with UVC, kill bedbugs.

2. The method of claim 1, further comprising generating hydroxyl radicals by irradiating UVC photons onto the area to be cleaned.

3. The method of claim 1, wherein the step of irradiating UVC photons onto the area to be cleaned is performed in a dark environment.

4. The method of claim 1, further comprising generating carbon dioxide, from oxidizing organic matter, to force bedbugs from hiding places on the area to be cleaned.

5. The method of claim 1, further comprising vacuuming the area to be cleaned.

6. The method of claim 1, further comprising repeating the steps of applying titanium dioxide and irradiating UVC photons onto the area to be cleaned.

7. The method of claim 5, further comprising steam cleaning an item having the area to be cleaned after the step of vacuuming the area to be cleaned.

8. A method for removing bedbugs, comprising:
applying titanium dioxide to an area to be cleaned;
irradiating ultraviolet C (UVC) photons onto the area to be cleaned, the UVC photons initiating a photo catalytic oxidation (PCO) process to kill bedbugs;
generating hydroxyl radicals by irradiating UVC photons onto the area to be cleaned;
generating carbon dioxide to force bedbugs from hiding places on the area to be cleaned; and
vacuuming the area to be cleaned.

9. The method of claim 8, further comprising steam cleaning an item having the area to be cleaned after the step of vacuuming the area to be cleaned.

10. The method of claim 8, wherein the step of irradiating UVC photons onto the area to be cleaned is performed in a dark environment.

11. The method of claim 8, further comprising repeating the steps of applying titanium dioxide and irradiating UVC photons onto the area to be cleaned.

12. A method for cleaning surfaces, the method comprising:
applying titanium dioxide to an area to be cleaned;
irradiating ultraviolet C (UVC) photons onto the area to be cleaned, the UVC photons initiating a photo catalytic oxidation (PCO) process to kill bedbugs; and
vacuuming the area to be cleaned.

13. The method of claim 12, further comprising generating hydroxyl radicals by irradiating UVC photons onto the area to be cleaned.

14. The method of claim 12, wherein the step of irradiating UVC photons onto the area to be cleaned is performed in a dark environment.

15. The method of claim 12, further comprising generating carbon dioxide to force bedbugs from hiding places on the area to be cleaned.

16. The method of claim 12, further comprising repeating the steps of applying titanium dioxide and irradiating UVC photons onto the area to be cleaned.

17. The method of claim 12, further comprising steam cleaning an item having the area to be cleaned after the step of vacuuming the area to be cleaned.
